(19) United States
(12) Patent Application Publication
(10) Pub. No.: US 2009/0208513 A1
(43) Pub. Date: Aug. 20, 2009

(54) Diagnostic And Therapeutic Uses Of Augmenter Of Liver Regeneration In Inflammatory Conditions

(75) Inventors: Chanrashekhar R. Gandhi, Pittsburgh, PA (US); Yoram Vodovotz, Sewickley, PA (US)

Correspondence Address: BAKER BOTTS L.L.P., 30 ROCKEFELLER PLAZA, 44TH FLOOR, NEW YORK, NY, 10112-4498, US

(73) Assignee: University of Pittsburgh - of The Commonwealth System Of Higher Education, Pittsburgh, PA (US)

(21) Appl. No.: 12/205,417

(22) Filed: Sep. 5, 2008

Related U.S. Application Data
(60) Provisional application No. 60/970,656, filed on Sep. 7, 2007.

(51) Int. Cl. A61K 39/395; C12Q 1/68; G01N 33/53; A61P 29/00

(52) U.S. Cl. 424/172.1; 436/86; 435/7.92

(57) ABSTRACT

The present invention provides for methods and kits for detecting sepsis, trauma/hemorrhage or inflammation in a subject. It is based, at least in part, on the discovery that Augmenter of Liver Regeneration (“ALR”) is an early marker of these conditions. Accordingly, in other embodiments, the present invention provides for a method of treating sepsis, traumatic/hemorrhagic shock and inflammation by inhibiting ALR.

### CROSS-REFERENCE TO RELATED APPLICATIONS

This application claims priority to U.S. Provisional Application Ser. No. 60/970,856, filed on Sep. 7, 2007, which is incorporated by reference herein in its entirety for all purposes.

### GRANT INFORMATION

The subject matter of the present application was developed, at least in part, under a Veteran's Administration Merit Review Grant and National Institute of General Medical Sciences Grant No. RO1-GM-67240-04.

### 1. INTRODUCTION

The present invention provides for methods and kits for detecting sepsis, traumatic/hemorrhagic shock or inflammation in a subject. It is based, at least in part, on the discovery that Augmenter of Liver Regeneration (“ALR”) is an early marker of sepsis and shock associated with trauma/hemorrhage. Accordingly, in other embodiments, the present invention provides for a method of treating sepsis, traumatic/hemorrhagic shock and/or inflammation by inhibiting ALR.

### 2. BACKGROUND OF THE INVENTION

Augmenter of Liver Regeneration (“ALR”) was originally identified in regenerating rat and canine livers following partial hepatectomy. ALR was subsequently found to be constitutively expressed, in an inactive form, in hepatocytes, then released from the cells, in active form, in response to certain stimuli, such as partial hepatectomy (for a review, see Gandhi et al., 1999, Hepatology 29:1435-1445).

For a review of the history of the cloning and molecular characterization of ALR, see Gatzidon et al., 2006, World J. Gastroenterol. 12(31):4951-4958 (see also Francavilla et al., 1994, Hepatology 20:747-757; Hagiya et al., 1994, Proc. Natl. Sci. U.S.A. 91:8142-8146; Hagiya et al., 1995, Proc. Natl. Acad. Sci. 92:3076 and Giorda et al., 1996, Mol. Med. 2:97-108). The murine and human ALR genes have been cloned and mapped to chromosomes 17 and 16, respectively (Giorda et al., 1996, Mol. Med. 2:97-108, Yang et al., 1997, Sci China 6:642-647). Whereas it was originally believed that the ALR protein was comprised of approximately 125 aa, upstream translation initiation sites were later recognized, and longer forms of ALR identified. For example, a long form of rat ALR was identified which is a 198-amino acid residue protein having a molecular weight of approximately 22 kd. (Gandhi et al., 1999, Hepatology 29:1435-1445). Similarly, a longer variant of human ALR having a length of 205 amino acids has been identified (Lu et al., 2002, World J. Gastroenterol. 8:353-356). Accordingly, “short forms” of ALR having approximately 125 aa residues and “long forms” of ALR having approximately 200 amino acid residues are referred to in the art.

As regards ALR function, in addition to its originally recognized association with regeneration, it is interesting to note that ALR is homologous to yeast scERV1, which is involved in mitochondrial biogenesis and the cell cycle. The carboxy termini of scERV1 and mammalian ALR are more than 40 percent identical, but human ALR cannot substitute for scERV1 in yeast, suggesting that the amino terminus may confer cell-specific functionality (Hofhaus et al., 1999, Eur. J. Cell Biol. 78(5):349-356).

In addition to its role in regeneration, Polimeno et al., 2000, Dig. Liver Dis. 32(6):510-517 reports that ALR induces an increase in mitochondrial gene expression and enhanced cytochrome content and oxidative phosphorylation capacity of liver-derived mitochondria. Polimeno et al., 2000, Dig. Liver Dis. 32(3):217-225 observed that ALR reduced interferon gamma in liver-resident natural killer cells (both anti-inflammatory effects). Lisowsky et al., 2001, Dig. Liver Dis. 33(2):173-180 report that human and rat ALR are flavin-linked sulfhydryl oxidases that catalyze the formation of disulfide bonds. The crystal structure of recombinant rat ALR (short form) has been determined and shows the molecule to be a homodimer bearing a non-covalently linked FAD (Wu et al., 2003, Protein Sci. 12(5):1109-1118). Farrel and Thorpe, 2005, Biochemistry 44(5):1532-1541 suggest that ALR may not function as a sulfhydryl oxidase in the mitochondrial intermembrane, but rather may interact, via cytochrome c, with the respiratory chain. In their studies of the effect of sepsis on hepatic mitochondrial DNA, Sullman et al., Am J Respir Crit Care Med 167:570-579 reported that in a rat model of sepsis, where rats were treated with lipopolysaccharide (“LPS”), ALR mRNA levels increased over the first day post-LPS treatment, and decreased thereafter.

### 3. SUMMARY OF THE INVENTION

The present invention relates to the discovery that levels of Augmenter of Liver Regeneration (“ALR”) increase quickly and are sustained in experimental models of sepsis, septic shock, traumatic/hemorrhagic shock, and inflammation. Accordingly, ALR may be used, in a diagnostic context, as an early marker of sepsis and trauma/hemorrhage. Such an early marker may be valuable in many clinical situations, especially where a subject may be at increased risk for sepsis as a result of immunocompromise due to disease, age (e.g., neonates and the elderly), chemotherapy (e.g. in cancer treatment), pharmacologic immunosuppression (e.g. to prevent transplant rejection), or bone marrow transplant.

In related embodiments, ALR may be used, according to the invention, as an early marker of inflammation in settings other than shock. In addition, the present invention provides for kits which may be used in the detection of sepsis and/or inflammation in a subject.

Moreover, in view of the early rise of ALR in sepsis and inflammation, the present invention provides for methods of treating these conditions in which ALR is inhibited.

### 4. BRIEF DESCRIPTION OF THE FIGURES

FIG. 1. ALR levels at various time points after administration of LPS to rats, as determined by an ELISA assay.

FIG. 2. ALR levels at various time points after introduction of a gram negative bacteria-containing (circles) or control (squares) fibrin clot, as determined by an ELISA assay.

FIG. 3A-D. Levels of inflammatory mediators (A) TNF, (B) IL-10, (C) IL-6, and (D) nitric oxide reaction products (No. 5, No. 5), at various time points after implantation of an E. coli containing fibrin clot in rats.

FIG. 4. ALR levels in a mouse model of hemorrhagic shock, at various time points post-hemorrhage, as measured by ELISA assay. The hatched line indicates predictions of “damage/dysfunction” induced by inflammatory stimuli following hemorrhagic shock, using a mathematical model of acute inflammation (Chow et al, 2005, Shock 24:74).

FIG. 5. Hepatic ALR levels in rats at various time points after surgical creation of a portocaval shunt (“PCS”), as measured by ELISA.

FIG. 6A-D. Effect of ALR and LPS on cytokine release by Kupffer cells, in particular (A) TGF-β1, (B) TNF-α, (C) IL-6, and (D) IL-1β.

FIG. 7. ALR release by primary hepatocytes treated with LPS, as measured by ELISA.

FIG. 8A-C. Effect on hepatocytes of ALR depletion by antisense oligonucleotide. Rat hepatocyte cultures were either (A) untreated, or treated with either (B) scrambled control oligonucleotide or (C) antisense to ALR (“ALR-AS”). Representative images acquired at the start of the experiment and at about 11 hours (control), 12 hours (scrambled) and 5.5 hours (ALR-AS) are shown.

FIG. 9A-D. Levels of ALR and other cytokines in human trauma patients at various times post-hospitalization. (A) Levels of ALR in patient HR002. (B) ALR levels in patient HR004. (C) levels of cytokines in patient HR002 (IL-6 levels represented by a dark circle, IL-10 levels represented by a square, and TNF levels represented by a clear circle). (D) levels of cytokines in patient HR004 (IL-6 levels represented by a dark circle, IL-10 levels represented by a square, and TNF levels represented by a clear circle).

FIG. 10A-D. Levels of ALR and other cytokines in human trauma patients at various times post-hospitalization. (A) Levels of ALR in patient HR006. (B) ALR levels in patient HR008. (C) levels of cytokines in patient HR006 (IL-6 levels represented by a dark circle, IL-10 levels represented by a square, and TNF levels represented by a clear circle). (D) levels of cytokines in patient HR008 (IL-6 levels represented by a dark circle, IL-10 levels represented by a square, and TNF levels represented by a clear circle).

### 5. DETAILED DESCRIPTION OF THE INVENTION

For clarity of description, and not by way of limitation, the detailed description of the invention is divided into the following subsections:

(i) methods of detecting sepsis, traumatic/hemorrhagic shock, or inflammation;

(ii) kits; and

(iii) methods of treatment.

### 5.1 Methods of Detecting Sepsis or Inflammation

In a first set of embodiments, the present invention provides for a method of detecting sepsis or traumatic/hemorrhagic shock in a subject, comprising measuring the level of Augmenter of Liver Regeneration (“ALR”) in a sample collected from the subject, and comparing the level to a baseline value, wherein an elevation in the level of ALR relative to baseline is an indicator that the subject suffers from sepsis.

In a second set of embodiments, the present invention provides for a method of detecting inflammation in a subject, comprising measuring the level of ALR in a sample collected from the subject, and comparing the level to a baseline value, wherein an elevation in the level of ALR relative to baseline is an indicator that the subject suffers from inflammation.

The baseline level of ALR may either be determined essentially contemporaneously as the level in the subject under consideration, or may be predetermined. As non-limiting examples, the baseline level may be predetermined in the subject while in a healthy state, or may be predetermined or contemporaneously measured in a healthy control subject who is not the subject under consideration. The baseline level occurs in a sample comparable with the subject sample being tested; for example, where the sample being tested is a plasma sample, the baseline level is measured in a plasma sample.

The subject may be a human or non-human subject. Examples of non-human subjects include a mouse, a rat, a rabbit, a horse, a dog, a non-human primate, a pig, a cow, or a cat.

The sample may be any tissue or fluid from the subject, including, but not limited to, blood, serum, plasma, urine, sputum, saliva, semen, cerebrospinal fluid, menstrual fluid, joint fluid, peritoneal fluid, gastric fluid, amniotic fluid, feces, cyst fluid, etc.

Any method to determine the level of a protein may be used, including, but not limited to, a Western blot, a dot-blot, an assay for flavin-linked sulfhydryl oxidase activity, or an enzyme-linked immunosorbent assay (“ELISA”), or a bead-based immunodetection assay.

In preferred, non-limiting embodiments of the invention, an ALR level is measured by an ELISA assay. For example, such an assay may comprise (i) binding ALR in the sample to a first antibody, or fragment thereof, which binds to ALR, bound to a solid substrate; (ii) washing the solid substrate to remove components other than ALR, and then (iii) exposing the solid substrate (which, depending on whether or not ALR is present, is associated with antibody/ALR complex or unbound antibody) to a second antibody or fragment thereof, which also binds to ALR (and may have essentially the same antibody features (e.g., variable region) as the first antibody or may bind to a different region of ALR than the first antibody), said second antibody linked to a detectable compound. The detectable compound may be directly detectable (for example, radioactive or fluorescent) or indirectly labeled (e.g. the second antibody may be linked to a ligand, which, when exposed to a complex of the receptor for that ligand and a detector compound (e.g., a radioactive label, fluorescent label, enzyme), binds said complex and becomes detectable).

In a specific, non-limiting example, the ALR level may be measured using a method essentially as set forth in Gandhi et al., 1999, Hepatology 29:1435-1445, which may optionally (see below) be modified to use antibody reagent prepared toward an ALR species other than rat (e.g., an antibody directed toward human ALR). See section 7 below. 96-well ELISA plates may be coated with anti-ALR antibody (1 μg/well) in 50 μL sample dilution buffer (0.1 mol/L Na₂CO₃ [pH 9.0]) by overnight incubation at 4° C. The medium may then be removed and the plates washed several times (e.g. 4 times) with wash buffer (20 mmol/L Tris [pH 7.5] containing 0.5 mol/L NaCl and 0.05% Tween 20). After blocking the nonspecific sites with “Super Block” (Pierce Chemical Co.), the plates may be washed (e.g., 4 times) with the wash buffer. Samples or controls dissolved in sample dilution buffer (20 mmol/L Tris [pH 7.5] containing 0.5 mol/L NaCl, 0.05% Tween 20) and 1% “Sea Block” (Pierce Chemical Co.) may then be added to the wells, and the plates incubated at 4° C. overnight. The plates may then be washed (e.g., 6 times), incubated with biotinylated anti-ALR antibody (for example, prepared using a kit from Vector Laboratories, Burlingame, Calif.) (0.2 μg/well in 100 μL sample dilution buffer) for 1 hour at room temperature, and washed (e.g., 6 times). The plates may then be incubated with avidin alkaline phosphatase (Sigma Chemical Co.) (100 μL/well of 1:1,000; vol/vol in sample dilution buffer) for 30 minutes at room temperature. After washing (e.g. 8 times), incubation may be performed in the dark with alkaline phosphatase substrate (Sigma Chemical Co.) (1 mg/ML in 0.1 mol/L NaHCO₃ containing 1 mmol/L MgCl₂; 100 mL/well, and the developed color may be read in a microplate spectrophotometer at 405 nm.

An antibody which may be used to detect an ALR level in a subject may be directed, in non-limiting embodiments, to a human long-form ALR (GenBank Accession No. NM_{—}005262; and see Giorda et al., 1996, Mol. Med. 2(1):97-108 and SEQ ID NO:2), a rat long-form ALR (Giorda et al., 1996, Mol. Med. 2(1):97-108), or a mouse long-form ALR (GenBank Acc. NO. BC023941; Giorda et al., 1996, Mol. Med. 2(1):97-108; GenBank Acc. No. U40496).

In specific non-limiting embodiments, an antibody for use in measuring levels of ALR may be directed to the following regions of ALR: residues MAAPS (SEQ ID NO: 5) through KDGSCD (SEQ ID NO:6) of the rat long-form ALR sequence (Giorda et al., 1996, Mol. Med. 2(1):97-108) or corresponding residues MAAPG (SEQ ID NO:7) through KDGSCD (SEQ ID NO:6)] of the human long-form sequence (SEQ ID NO:2), or an antibody may be directed to (e.g., produced using as immunogen) MRTQQKRTKFRED (SEQ ID NO:3). Generation of antibodies may be by any method known in the art. In a specific non-limiting embodiment, for example, rabbits (approximately 3 kg) may be prebled and immunized with intradermal injections of an adjuvant (e.g., complete or incomplete Freund's adjuvant). Three weeks after the initial immunization and every 3 weeks thereafter, mixtures of the conjugates of keyhole limpet hemocyanin (KLH) with rrALR (250 μg) or its peptide fragments (250 μg) in phosphate buffered saline (PBS) may be injected subcutaneously at multiple sites. Ten to 12 days after each immunization, approximately 20 mL blood may be drawn, and serum may be separated by centrifugation. For isolation of the IgG fraction, the serum may be diluted with 10 volumes of 10 mmol/L sodium borate (pH 8.0) and loaded on a Protein A column equilibrated in the same buffer. The column may be washed until the absorbance of the eluted fractions at 280 nm is similar to that of the borate buffer, and the IgG may be eluted with Pierce gentle elution buffer (Pierce Chemical Co. Rockford, Ill.). Fractions with absorbance greater than 0.05 as compared with the elution buffer may be pooled and dialyzed against two changes of Tris-buffered saline buffer in a 1,000-mw cut-off SpectraPor6 dialysis tubing (Spectrum Industries).

The method of determining the level of ALR may utilize test reagents specific for a given species or may use a test reagent that may cross-react across species (e.g., an antibody molecule).

### 5.2 Kits

In non-limiting embodiments, the present invention provides for a kit for detecting sepsis or inflammation in a subject, comprising (i) a first antibody, or fragment thereof, which binds to ALR, optionally bound to a solid substrate (e.g., a multi-well culture plate); (ii) a second antibody, which also binds to ALR, said second antibody linked to a detectable compound; optionally (iii) a means for measuring the level of an inflammatory mediator and optionally (iv) instructions that include a description of the inventive methods of using ALR levels to diagnose sepsis, shock, and/or inflammation.

The detectable compound may be directly detectable (for example, radioactively or fluorescently labeled) or indirectly labeled (e.g. the second antibody may be linked to a ligand, which, when exposed to a complex of the receptor for that ligand and a detector compound (e.g., a radioactive label, fluorescent label, enzyme), binds to the complex and becomes detectable). In various non-limiting embodiments, the detectable compound linked to the second antibody is a ligand and the kit further comprises a detector linked to a receptor for the ligand. For example, the detector may be an enzyme and the kit may further comprise a substrate for the enzyme, wherein the product of action of the enzyme on the substrate is detectable. As a specific, non-limiting example, the ligand may be biotin and the detector may comprise avidin (or vice-versa). As a specific, non-limiting example, the detector may comprise alkaline phosphatase enzyme.

In preferred, specific embodiments, an antibody for use in measuring levels of ALR may be directed to the following regions of ALR: residues MAAPS (SEQ ID NO:5) through KDGSCD (SEQ ID NO:6) (Giorda et al., 1996, Mol. Med. 2(1):97-108) of the rat long-form ALR sequence or corresponding residues MAAPG (SEQ ID NO:7) through KDGSCD (SEQ ID NO:6) of the human long-form sequence, or an antibody may be directed to (e.g., produced using as immunogen) MRTQQKRTKFRED (SEQ ID NO:3).

### 5.3 Methods of Treatment

In particular non-limiting embodiments, the present invention provides for a method of treating sepsis or traumatic/hemorrhagic shock, comprising administering, to a subject suffering from sepsis, an effective amount of an inhibitor of ALR.

In other particular non-limiting embodiments, the present invention provides for a method of treating inflammation, comprising administering, to a subject suffering from inflammation, an effective amount of an inhibitor of ALR.

In certain non-limiting embodiments of the invention, the inhibitor of ALR may be an antibody. In specific, non-limiting embodiments, an antibody for use in the foregoing methods may be directed to the following regions of ALR: residues MAAPS (SEQ ID NO:5) through KDGSCD (SEQ ID NO:6) (Giorda et al., 1996, Mol. Med. 2(1):97-108) of the rat long-form ALR sequences or corresponding residues MAAPG (SEQ ID NO:7) through KDGSCD (SEQ ID NO:6) of the human long-form sequence, or an antibody may be directed to (e.g., produced using as immunogen) MRTQQKRTKFRED (SEQ ID NO:3).

In other non-limiting embodiment, the inhibitor of ALR may be a nucleic acid, for example, an antisense nucleic acid, an interfering RNA, or a catalytic nucleic acid. Such molecules may preferably comprise a region which is complementary to one of the ALR genes set forth above, which is at least about 5-30 bp, or about 10-25 bp, in length. In a specific, non-limiting embodiment, the molecule is an antisense molecule having the sequence 5′GACTGCCGCGAGGGAAACCT 3′ (SEQ ID NO:1). In particular non-limiting embodiments, said nucleic acids may comprise nucleotides modified to enhance stability, such as, for example, phosphorothioated nucleotides. Said nucleic acids may comprise ribonucleotides, deoxyribonucleotides, or both. The amount of said nucleic acid inhibitor of ALR may, in preferred but non-limiting embodiments, be limited so as to reduce the level of ALR to within normal limits, as ALR has been shown to be important intracellular factor for maintaining hepatocyte viability (see, for example, section 6 below as well as Thirunavukkarasu et al., 2008, J. Hepatol., 48:578-588).

The ALR inhibitor may be administered by any standard method or route, including but not limited to local administration to an affected tissue (e.g., injection or instillation into an inflamed tissue or area (e.g., a joint space), or topical application), systemic injection intravenously or intraarterially, intraperitoneal administration, intrathecal administration, intramuscular injection, subcutaneous injection, inhalation, etc.

### 6. WORKING EXAMPLE

### ALR is an Early Indicator of Sepsis

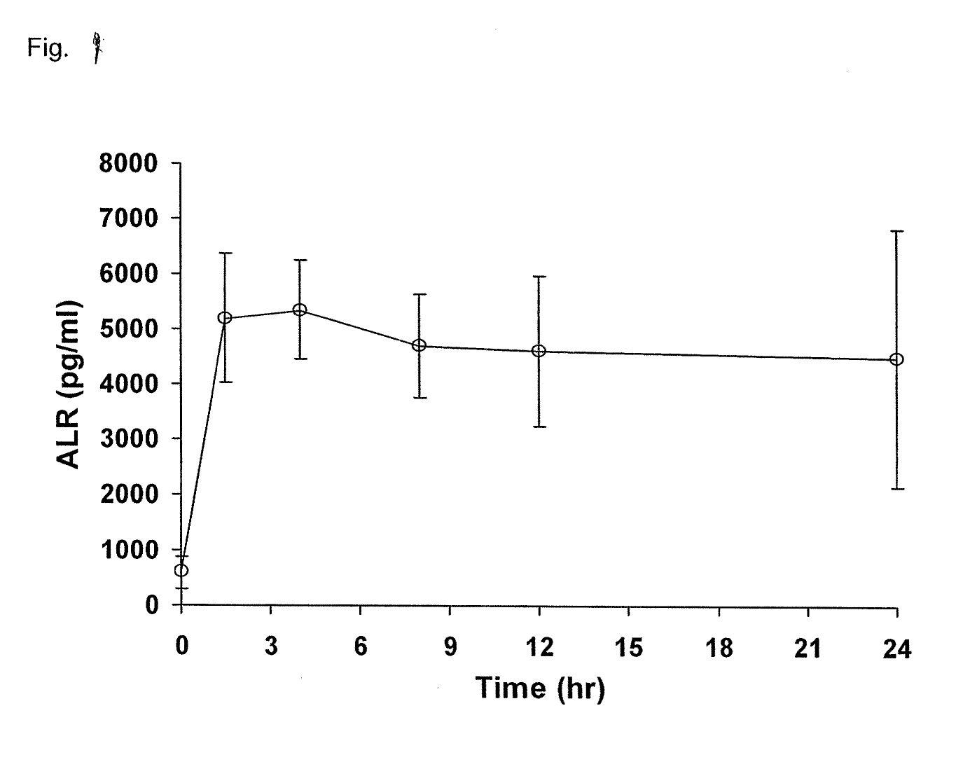

In a first set of experiments, the levels of ALR were measured in a rat model of endotoxemia. Rats (Sprague-Dawley, n=4 per time point) received 6 mg/kg LPS from E. coli O111:B4 intraperitoneally. At various time points following this injection, they were euthanized by exsanguination under excess inhalation anesthesia and plasma obtained for measurement of ALR. ALR was extracted and its concentration determined by ELISA essentially as described previously (Gandhi et al., 1999, Hepatology 29:1435-1445). As shown in FIG. 1, ALR was found to be persistently elevated starting at early time points following injection of LPS.

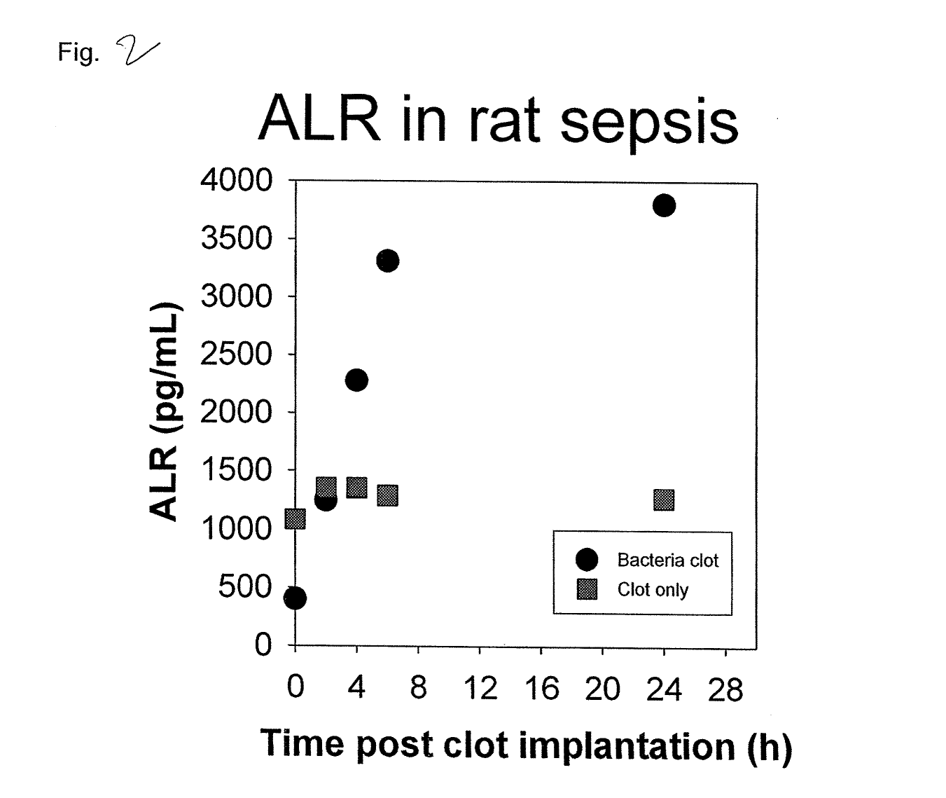

Next, the levels of ALR were determined in a rat model of gram-negative sepsis. Sprague-Dawley rats (one per time point) were subjected to surgical implantation of a fibrin clot containing 1.5×10⁸ E. coli strain E25922 or a sham fibrin clot containing no bacteria. At various time points after the implantation procedure, the rats were euthanized by exsanguination under excess inhalation anesthesia and plasma obtained for measurement of ALR. ALR was extracted and its concentration determined by ELISA (Gandhi et al., 1999, Hepatology 29:1435-1445). As depicted in FIG. 2, ALR was observed to be elevated persistently in response to true bacterial sepsis.

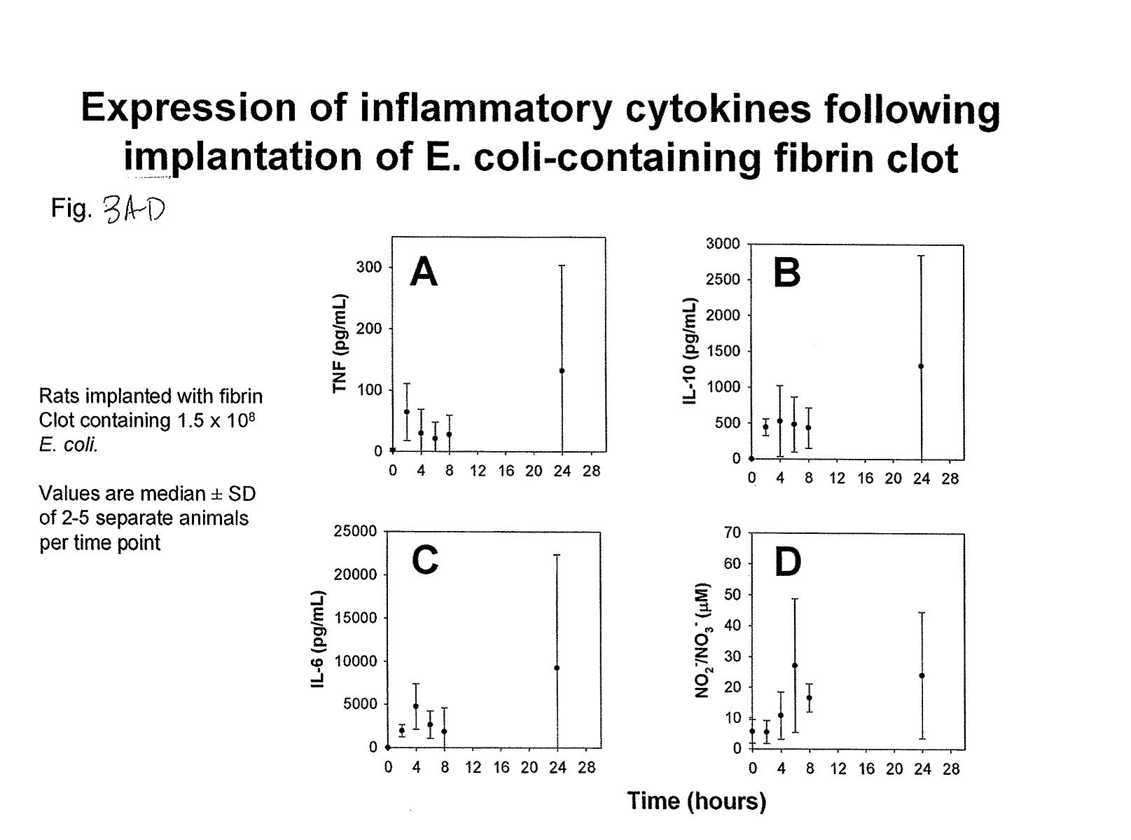

To evaluate the timing of the ALR response with that of other cytokines, the levels of other cytokines was measured in the same sepsis model described above. Plasma samples from the same rats described in the preceding paragraph were assayed for tumor necrosis factor-α (TNF), interleukin (IL)-6, IL-10, and the nitric oxide (NO) reaction products NO₂⁻/NO₃⁻ using commercially available kits, and the results are shown in FIG. 3A-D. These results, compared to FIG. 2, show that in contrast to ALR, other markers of inflammation are not persistently increased in the first 8 hours.

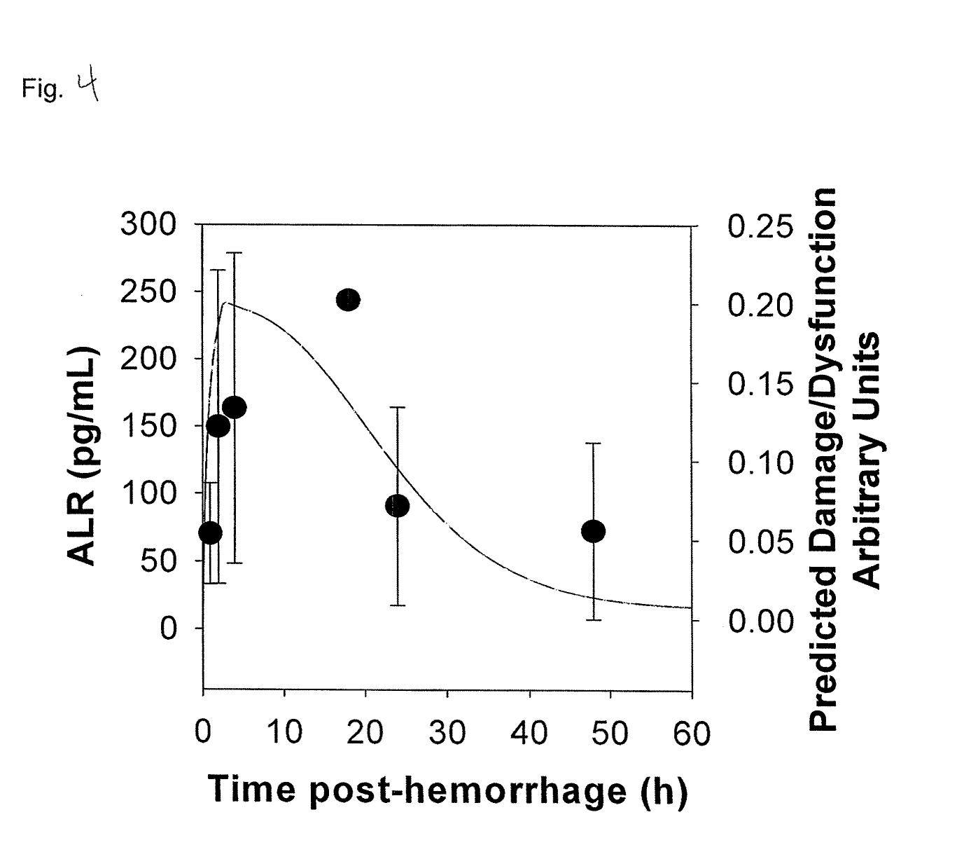

Next, ALR levels were determined in a mouse model of surgical cannulation followed by hemorrhagic shock (reduction of mean arterial pressure to 25 mmHg for 2.5 h, followed by resuscitation and observation for the time points indicated). Mice (C57B1/6; 1-4 per time point) were cannulated and subjected to hemorrhagic shock (bleeding to a mean arterial pressure of 25 mmHg for 2.5 hours), followed by resuscitation at the indicated time points. The mice were euthanized by exsanguination under excess inhalation anesthesia and plasma obtained for measurement of ALR. ALR was extracted and its concentration determined by ELISA essentially as described previously (Gandhi et al., 1999, Hepatology 29:1435-1445). The results are shown in FIG. 4. The hatched line indicates predictions of “damage/dysfunction” induced by inflammatory stimuli following hemorrhagic shock, using a mathematical model of acute inflammation (Chow et al, Shock, 2005. 24:74). ALR is seen to be elevated and to return towards baseline values following hemorrhagic shock in mice. The time course of ALR closely approximates that predicted for “damage/dysfunction” in the mathematical model. The “damage/dysfunction” variable in this model represents “alarm/danger” signals (Gallucci and Matzinger, 2001, Curr. Opin. Immunol. 13(1): 14-119).

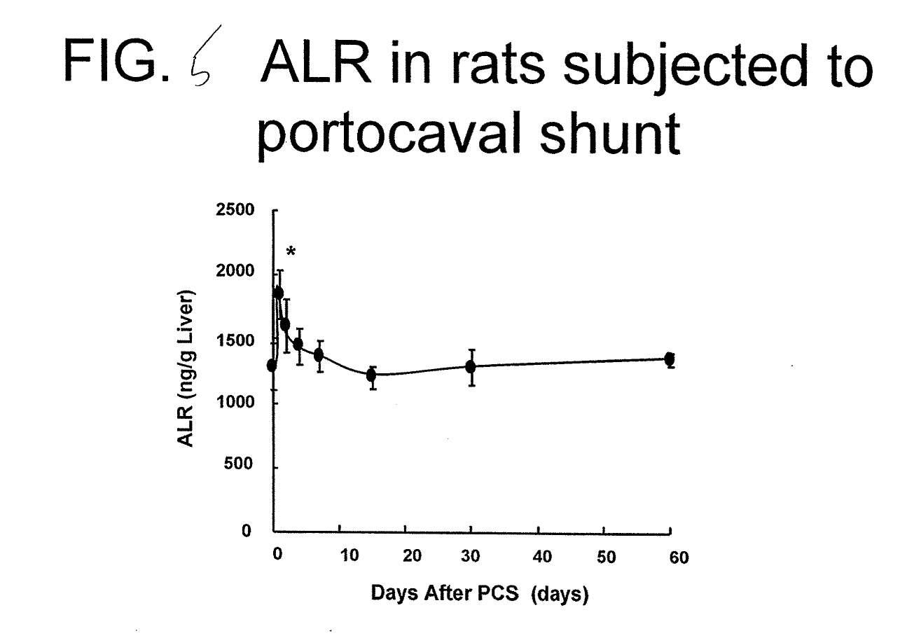

As shown in FIG. 5, ALR levels were found to increase rapidly after portacaval shunt surgery (a procedure that results in an inflammatory response) in rat. The surgical procedure was performed on preweighed Sprague-Dawley rats under methoxyflurane anesthesia. Laparotomy was performed and the hepatoesophageal plexus was ligated. Side-to-side anastomosis was created between the portal vein and inferior vena cava using 10-0 Novafil suture as described by Lee and Fisher, 1961, Surgery 50:668-672. After the shunt was examined to ensure its patency, the portal vein was carefully separated from hepatic artery and ligated at the hepatic hilum to create total PCS. One, 2, 4, 7, 15, 30 and 60 days after the creation of PCS, rats were sacrificed (three for each time point). The liver was excised after laparotomy, weighed, rinsed in ice-cold phosphate-buffered saline, snap-frozen in liquid nitrogen, crushed and saved in portions at −80° C. ALR was extracted and its concentration determined by ELISA essentially as described previously (Gandhi et al., 1999, Hepatology 29:1435-1445). ALR protein increased by 44% at 24 h before gradually declining to the basal value by day 15 suggesting that the liver injury causes early increase in the synthesis of ALR. For details, see Gandhi et al., Journal of Hepatology 37 (2002) 340-348.

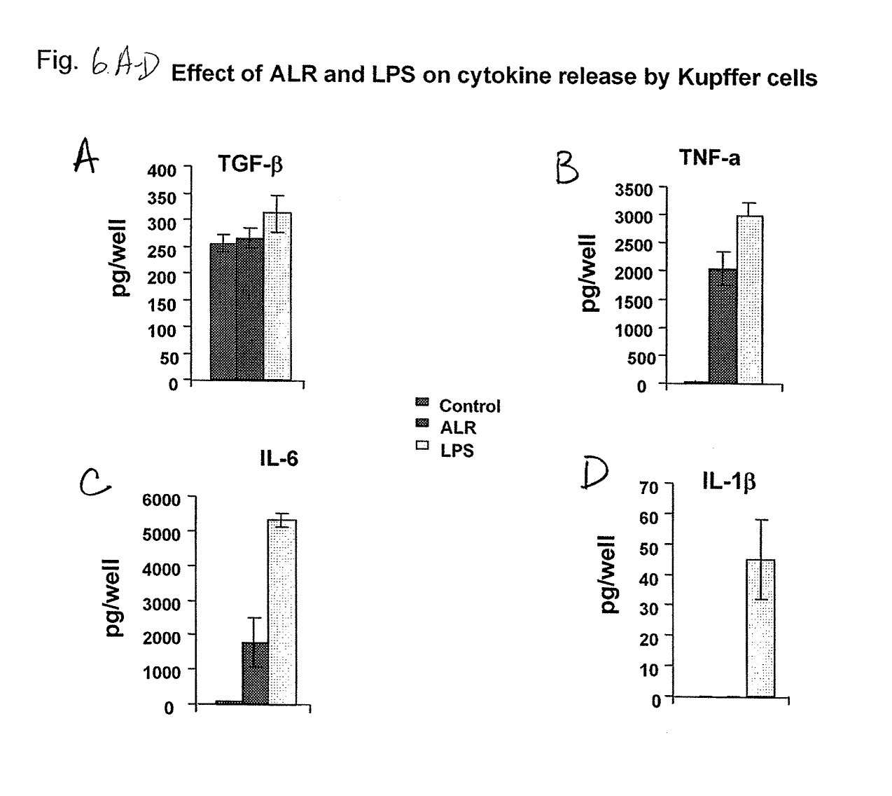

FIG. 6A-D shows the effect of ALR on cytokine synthesis in cultured Kupffer cells. Kupffer cells were prepared from livers of male Sprague-Dawley rats as described (Gandhi et al., J Biol Chem 1990; 265: 18234-18241). The cells were placed in culture on plastic dishes and stimulated with 1 μg/ml ALR or 1 μg/ml LPS. At 24 hours, various cytokines in the culture supernatant were measured by ELISA. ALR stimulated TNF-α and IL-6 synthesis and release (but not IL-1β) from Kupffer cells, while LPS stimulates the synthesis of all cytokines. Both substances did not stimulate TGF-β synthesis. These data suggest that the early-appearing ALR may be an inducer of inflammatory cytokines, in contrast to findings reported in Polimeno, et al., 2000, Dig. Liv. Dis. 32(3): 217-225).

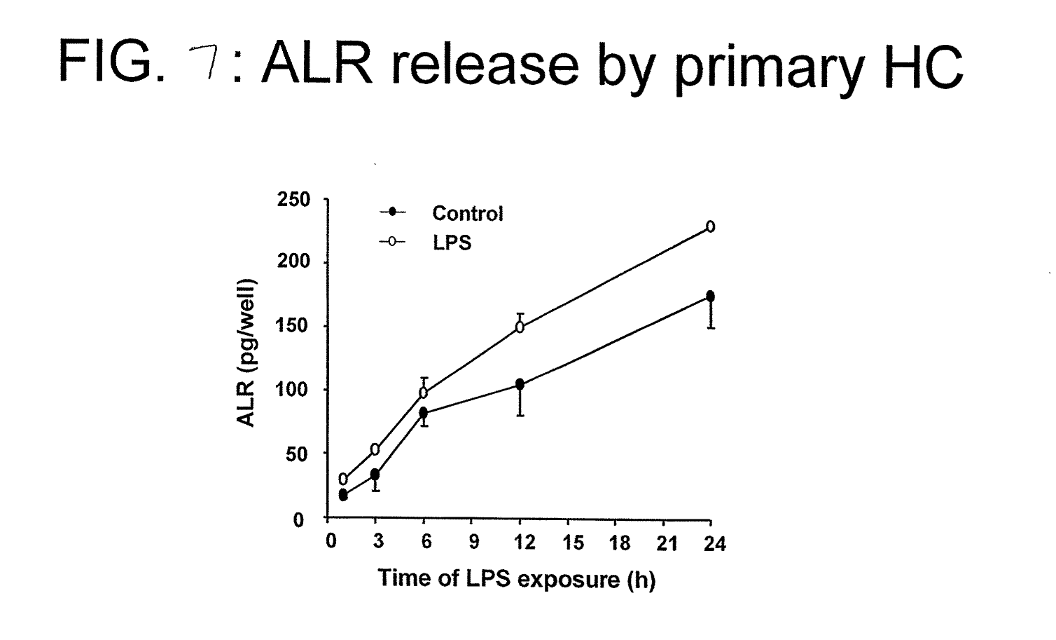

To evaluate the effect of sepsis on a cellular level, the effect of LPS on the release of ALR from hepatocytes was evaluated. Hepatocytes obtained from male Sprague-Dawley rats (Uemura et al., 2001, Br J Pharmacol 133:1125-1133) were placed in primary culture and stimulated with 1 μg/ml LPS in serum-free condition. At various time points, ALR levels in the culture supernatant were measured by ELISA as described (Gandhi et al., 1999, Hepatology 29:1435-1445). As shown in FIG. 7, the release of ALR was augmented by LPS at all time points as compared to the control.

In view of the foregoing results, which indicate that ALR may be an inducer of other cytokines in the context of stressors such as toxin, infection, and hypoxia, it may be postulated that ALR has the properties of an alarm/danger signal: a protein that mediates a central cellular function which, in settings of stress and inflammation, is released into the circulation and induces further inflammation (Gallucci and Matzinger, 2001, Curr. Opin. Immunol. 13 (1):114-119). In this regard, ALR may be compared to another protein, high mobility group box-1 (HMGB-1), a DNA binding protein that is released in settings of inflammation (Yang et al., 2005, J. Leukoc. Biol. 78 (1):1-8), is a central mediator of sepsis lethality (Wang et al., 1999, Science 285 (5425):248-251) and trauma/hemorrhage-induced injury (Fan et al., 2007, J. Immunol. 178:6573-6580), and which causes the production of cytokines such as tumor necrosis factor-alpha.

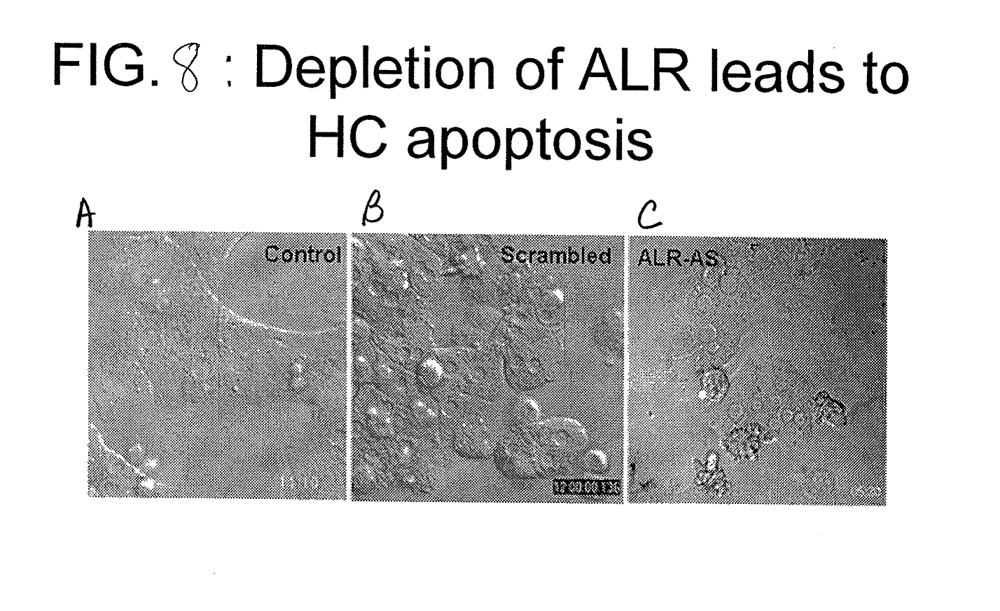

To demonstrate the role of ALR in normal cell function, the effect of ALR depletion was studied in hepatocyte culture. Hepatocytes in primary culture were plated on 40-mm collagen coated (10%) coverslips, and transfected with antisense-ALR oligonucleotide or scrambled oligonucleotide; control cells were left untreated. The sequence of phosphorothioated ALR-AS-oligonucleotide complementary to the nucleotide sequence 24 to 43 downstream from the start codon of ALR mRNA was 5′GACTGCCGCGAGGGAAACCT 3′ (SEQ ID NO: 1). The sequence of the scrambled oligonucleotide was 5′ACTGACAGATCGGGCAAGCC 3′ (SEQ ID NO:4). Five bases of the oligonucleotides at both 5′ and 3′ ends were replaced with 2′-O-Meth RNA, and internal dCs were replaced with 5-Me-dC. The oligonucleotides were FAM-labeled at the 5′ end and a hairpin loop was introduced at the 3′ end. Lipofectamine (8 μl/ml; Invitrogen) and the oligonucleotides (final concentration 1.2 nM) in lipofectamine were incubated separately in 200 μl of Opti-MEM medium (GibcoBRL, Grand Island, N.Y.) for 30 min. 800 μl of opti-MEM was added and the combined solution was overlaid onto the cells. The slides were inserted in a FCS2 closed heated (37° C.) live cell chamber (Bioptics: Bulter, Pa.), and the media were perfused through the chamber with a syringe pump at a rate of 0.5 ml/h. The FCS2 chamber was placed on a Nikon TE300 inverted microscope (Melville, N.Y.). Using a 40× dry objective, five stage positions were imaged with transmitted light every three minutes for approximately 18 h. The images were acquired and processed with Metamorph software (Universal Imaging Corporation, Downingtown, Pa.). Images of control, and scrambled (Scr)- or ALR-AS-treated hepatocytes were acquired every 3 min. Representative images acquired at the start of the experiment and at about 11 hours (control), 12 hours (scrambled) and 5.5 hours (ALR-AS) are shown in FIG. 8A-C. Clearly, the ALR-AS-treated cells show classical evidence of apotosis (a form of programmed cell death, the hallmarks of which are cell shrinkage and membrane blebbing). Thus, ALR is necessary for normal cellular function (survival). FIG. 6, in turn, shows that treatment of liver inflammatory cells (Kupffer cells) with ALR leads to the production of inflammatory cytokines, thereby demonstrating the other characteristic of an alarm/danger signal.

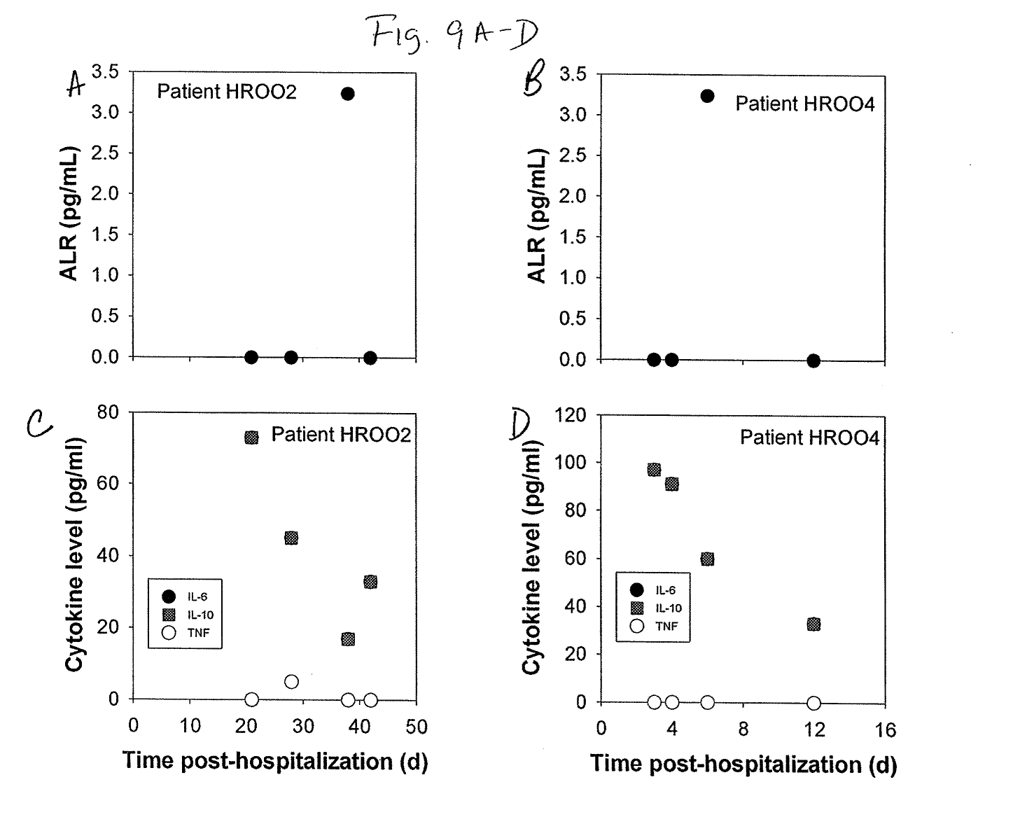

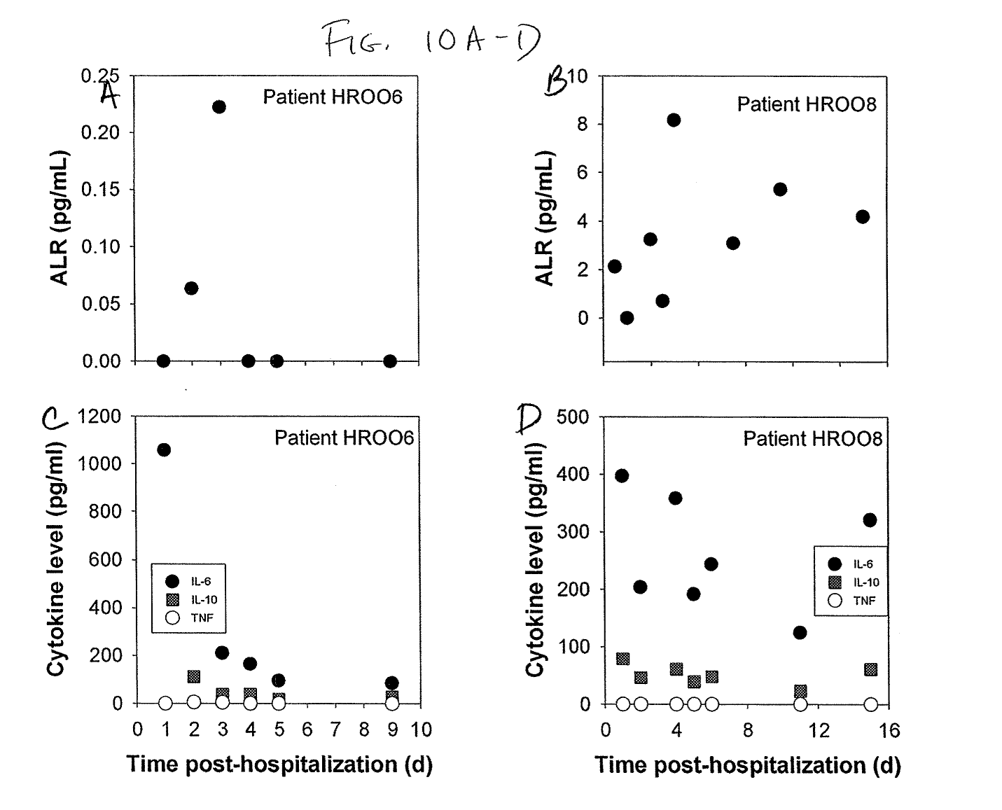

Finally, levels of ALR and the cytokines IL-6, IL-10 and TNF-α were evaluated at various time points in human trauma patients. Human trauma patients were sampled daily following trauma and admission to University of Pittsburgh Medical Center hospital, following approval from the University of Pittsburgh's Institutional Review Board. ALR was extracted and its concentration determined by ELISA essentially as described previously (Gandhi et al., 1999, Hepatology 29:1435-1445). The cytokines TNF, IL-6, and IL-10 were assayed using specific ELISA's. The data, presented in FIG. 9A-D and FIG. 10A-D, show diverse patterns of circulating ALR post-trauma.

### 7. WORKING EXAMPLE

### Elisa Assay

(as published in Gandhi et al., 1999, Hepatology 29:1435-1445)

ALR ELISA. Immulon-1 flat bottom 96-well ELISA plates (Dynatech Labs, Chantilly Va.) were coated with anti-rrALR antibody (1 μg/well) in 50 μL sample dilution buffer (0.1 mol/L Na₂CO₃ [pH 9.0]) by overnight incubation at 4° C. The medium was removed and the plates washed (4×) with the wash buffer (20 mmol/L Tris [pH 7.5] containing 0.5 mol/L NaCl and 0.05% Tween 20). After blocking the nonspecific sites with “Super Block” (Pierce Chemical Co.), the plates were washed (4×) with the wash buffer. Samples or standards dissolved in sample dilution buffer (20 mmol/L Tris [pH 7.5] containing 0.5 mol/L NaCl, 0.05% Tween 20) and 1% “Sea Block” (Pierce Chemical Co.) were added to the wells, and the plates incubated at 4° C. overnight. The plates were washed (6×), incubated with biotinylated anti-rrALR antibody (prepared using a kit from Vector Laboratories, Burlingame, Calif.) (0.2 μg/well in 100 μL sample dilution buffer) for 1 hour at room temperature, and washed (6×). The plates were incubated with avidin alkaline phosphatase (Sigma Chemical Co.) (100 μL/well of 1:1,000; vol/vol in sample dilution buffer) for 30 minutes at room temperature. After washing (8×), incubation was performed in the dark with alkaline phosphatase substrate (Sigma Chemical Co.) (1 mg/ML in 0.1 mol/L NaHCO₃ containing 1 mmol/L MgCl₂; 100 mL/well, and the developed color was read in a microplate spectrophotometer at 405 nm.

### 8. WORKING EXAMPLE

### Preparation of Antibody

Antibodies were prepared against rat recombinant ALR (rrALR) and its peptide fragments (Covance, Inc., Denver, Pa.). Briefly, white New Zealand female rabbits (approximately 3 kg) were prebled and immunized with intradermal injections of the adjuvant. Three weeks after the initial immunization and every 3 weeks thereafter, mixtures of the conjugates of keyhole limpet hemocyanin with rrALR (250 μg) or its peptide fragments (250 μg) in phosphate buffered saline (PBS) were injected subcutaneously at multiple sites. Ten to 12 days after each immunization, approximately 20 mL blood was drawn, and serum was separated by centrifugation. For isolation of the IgG fraction, the serum was diluted with 10 volumes of 10 mmol/L sodium borate (pH 8.0) and loaded on a Protein A column (Pharmacia Biotechnology) equilibrated in the same buffer. The column was washed until the absorbance of the eluted fractions at 280 nm was similar to that of the borate buffer, and the IgG was eluted with Pierce gentle elution buffer (Pierce Chemical Co. Rockford, Ill.). Fractions with absorbance greater than 0.05 as compared with the elution buffer were pooled and dialyzed against two changes of Tris-buffered saline buffer in a 1,000-mw cut-off SpectraPor6 dialysis tubing (Spectrum Industries).

Various publications are cited herein, the contents of which are hereby incorporated by reference in their entireties.

We claim:

1. A method of detecting sepsis in a subject, comprising measuring the level of Augmenter of Liver Regeneration in a sample collected from a subject to be tested for sepsis, and comparing the level to a baseline value, wherein an elevation in the level of Augmenter of Liver Regeneration relative to baseline is an indicator that the subject suffers from sepsis.

2. The method of claim 1, where the level of Augmenter of Liver Regeneration is measured by an enzyme-linked immunosorbent assay.

3. A method of detecting inflammation in a subject, comprising measuring the level of Augmenter of Liver Regeneration in a sample collected from a subject to be tested for inflammation, and comparing the level to a baseline value, wherein an elevation in the level of Augmenter of Liver Regeneration relative to baseline is an indicator that the subject suffers from inflammation.

4. The method of claim 3, where the level of Augmenter of Liver Regeneration is measured by an enzyme-linked immunosorbent assay.

5. A method of detecting traumatic/hemorrhagic shock in a subject, comprising measuring the level of Augmenter of Liver Regeneration in a sample collected from a subject to be tested for inflammation, and comparing the level to a baseline value, wherein an elevation in the level of Augmenter of Liver Regeneration relative to baseline is an indicator that the subject suffers from inflammation.

6. The method of claim 5, where the level of Augmenter of Liver Regeneration is measured by an enzyme-linked immunosorbent assay.

7. A method of treating sepsis, comprising administering, to a subject suffering from sepsis, an effective amount of an inhibitor of Augmenter of Liver Regeneration.

8. A method of treating inflammation, comprising administering, to a subject suffering from inflammation, an effective amount of an inhibitor of Augmenter of Liver Regeneration.

9. A method of treating traumatic/hemorrhagic shock, comprising administering, to a subject suffering from inflammation, an effective amount of an inhibitor of Augmenter of Liver Regeneration.


SEQUENCE LISTING (7 sequences)

SEQ ID NO: 1 - DNA, 20 bp, Artificial Sequence
  Synthetic oligonucleotide
gactgccgcgagggaaacct

SEQ ID NO: 2 - protein, 205 aa, Homo sapiens
MAAPGERGRFHGGNLFFLPGGARSEMMDDLATDARGRGAGRRDAAASASTPAQAPTSDSPVAEDASRRRPCRACVDFKTWMRTQQKRDTKFREDCPPDREELGRHSWAVLHTLAAYYPDLPTPEQQQDMAQFIHLFSKFYPCEECAEDLRKRLCRNHPDTRTRACFTQWLCHLHNEVNRKLGKPDFDCSKVDERWRDGWKDGSCD

SEQ ID NO: 3 - protein, 13 aa, Artificial Sequence
  Synthetic polypeptide
MRTQQKRTKFRED

SEQ ID NO: 4 - DNA, 20 bp, Artificial Sequence
  Synthetic oligonucleotide
actgacagatcgggcaagcc

SEQ ID NO: 5 - protein, 5 aa, Rat (Rattus)
  UNSURE (1)...(5): 5 amino acid residue fragment of the rat ALR polypeptide
MAAPS

SEQ ID NO: 6 - protein, 6 aa, Unknown
  6 amino acid residue fragment of both the rat and human ALR polypeptide
KDGSCD

SEQ ID NO: 7 - protein, 5 aa, homo sapiens
  UNSURE (1)...(5): 5 amino acid residue fragment of the human ALR polypeptide
MAAPG